(19) United States
(12) Patent Application Publication
(10) Pub. No.: US 2020/0093990 A1
(43) Pub. Date: Mar. 26, 2020

(54) METHOD AND DEVICE FOR INTRODUCING AT LEAST ONE QUANTITATIVELY DETERMINED EXOGENOUS SUBSTANCE INTO AN ENDOGENOUS LIQUID

(71) Applicant: MEDICAL SCIENTIFIC INNOVATIONS - MSI, Paris (FR)

(72) Inventors: Pasem AHMED, Gennevilliers (FR); Pierre CHASTANIER, Paris (FR); Olivier BAILLIART, Paris (FR); Raymond POUGET, Boulogne-Billancourt (FR); Michel DIXMIER, Paris (FR); Mohand Boujema BRAHIMI, Paris (FR)

(21) Appl. No.: 16/487,260

(22) PCT Filed: Feb. 21, 2018
(86) PCT No.: PCT/FR2018/050409

§ 371 Date: Nov. 21, 2019

(30) Foreign Application Priority Data
Feb. 22, 2017 (EP) 17157500.4

(51) Int. Cl. A61M 5/172; A61B 5/15; A61B 5/1455; A61B 5/00; A61B 5/145

(52) U.S. Cl. CPC A61M 5/1723; A61B 5/150992; A61B 5/1455; A61M 2230/20; A61B 5/14546; A61M 2005/1726; A61M 2205/3313; A61B 5/742

(57) ABSTRACT

The present invention relates to a device for recognising, measuring and quantitatively determining at least one exogenous substance in an endogenous liquid. According to the invention, the device comprises: —a means for suctioning (1) an endogenous liquid containing at least one exogenous substance; —a means for collecting (2) the endogenous liquid containing at least one exogenous substance; —a spectrometer (3), preferably a Raman or infrared spectrometer, capable of acquiring and analysing said collected liquid; —a database-type data storage means comprising spectra relating to at least one exogenous substance; —a means for comparison between the spectrum or spectra acquired and the spectrum or spectra of the database; —a means (4) for controlled introduction of at least one exogenous substance into the endogenous liquid, as a function of the result of said comparison. The invention also relates to the method implemented by such a device.

### TECHNICAL FIELD OF THE INVENTION

The invention relates to the field of dosing an exogenous substance into an endogenous liquid. More specifically, the invention targets the measurement and the controlled introduction of an exogenous substance such as an antibiotic into an endogenous liquid such as blood. A virtually instantaneous controlled introduction or dose is targeted by the invention.

### PRIOR ART

As is known, the administration of exogenous substances such as antibiotics into the blood is carried out by intermittent perfusion or else by continuous perfusion. It is entirely necessary to monitor the “serum” concentrations of said antibiotics.

Currently, these assays are carried out by apparatus based on mass spectrometry, with methods which are expensive and are therefore not carried out frequently. A sample of blood is taken, which is analyzed for example with a Raman spectrometer then, as a function of the result, a determined dose of antibiotics is injected into the patient. This operation is carried out for example every four hours.

In the case of septic shock, it is necessary to administer the antibiotic(s) in the hour following the shock; unfortunately this is not always possible or simple.

In order to characterize and assay the samples, use may be made of Raman spectroscopy or else infrared spectroscopy. Regarding infrared spectroscopy, one of the recurrent problems lies in the choice of the wavelength; this is because the absorption bands are broad and overlap. It is possible to identify pure compounds or impurities; for the majority of organic compounds; the spectrum comprises several bands corresponding to several criteria which can thus be used as long as there is no overlap with the absorption peaks of other substances present in the sample.

It is known to use infrared spectroscopy for both qualitative and quantitative analyses in the fields of pharmacy, agriculture and food industries. In the pharmaceutical industry it is common to use methods based on infrared spectroscopy to assay the active ingredients of tablets; in agriculture, it is used for example to determine the content of crude protein contained in dry hay silage. In the food industry, it is used for assaying fats, proteins and lactose in milk or else to monitor, in a non-destructive manner, the contents of ethanol in fermentation processes. This investigative method has the advantage of being non-destructive and non-invasive. However, aside from the disadvantages already mentioned, this method requires a set of samples for calibration, preferably obtained from real samples in which the constituents to be analyzed are at sufficiently different concentrations.

Raman spectroscopy, the physical principle of which will not be recalled here, has been used since the end of the 1980s. It is worth noting that this method, like infrared spectrometry, is non-destructive and non-invasive. It is further easy to carry out and requires only a small amount of sample, of the order of a microgram. It can be used in a large number of cases: heterogeneous materials, or samples in which the structure may be oriented or not oriented. It may be coupled with other analytical methods, and offers the possibility of in situ measurements. It can be applied to atomic solids and can be used on samples of a very small size, down to 10⁻¹⁸ m³. This technique also makes it possible to work in hostile environments, especially at high temperature in the presence of radioactive phenomena or under controlled atmosphere. Moreover, this method is sensitive to small structures: identification of amorphous systems, analysis of very thin films for which scattering methods are sometimes difficult to carry out.

Thus, the possibility of rapidly analyzing a broad range of samples and the ability to collect a large number of spectra with high resolution in a single measurement have made the Raman technique accessible to a much more extensive number of scientific fields.

Portable apparatus using Raman spectroscopy, often used for the identification of cosmetic, pharmaceutical and chemical materials, are known. These devices especially enable the quick and precise identification of starting materials, the inspection of finished products and the detection of counterfeits. These portable Raman analyzers also make it possible to carry out quality controls, directly on-site and in a few seconds, on a wide variety of solids, liquids, powders and other substances. It is moreover possible to measure through transparent packaging, such as plastic bags and glass bottles.

As is known, infrared spectroscopy and Raman spectroscopy enable real-time analyses with portable apparatus, which is highly advantageous. By way of comparison, Raman spectroscopy makes it possible to obtain roughly the same type of results as infrared spectroscopy, even though the principle of the method is different. Raman spectroscopy is less used because of its cost but nonetheless has advantages over infrared spectroscopy.
Use of water, an excellent solvent for Raman spectroscopy. Indeed, the presence of water is unproblematic because water exhibits very limited Raman scattering;

Use of glass cuvettes: glass is transparent in the spectral ranges in question and the Raman spectrum thereof is very weak.

The samples may be used in any form, without being diluted or altered.

The spectra are generally simpler, such that band overlaps are much less frequent.

Possibility of studying entirely symmetrical vibrational modes, which are invisible in infrared.

Obtaining complementary information by light polarization.

The Raman effect is independent of the exciting wavelength used, which may make it possible to suppress certain undesirable phenomena (fluorescence, decomposition of colored substances, etc.) by choosing a suitable wavelength.

No permanent polarization of the molecules is necessary.

Use of a single apparatus and a single wavenumber scan to cover the entirety of the spectrum of vibration frequencies, due to the very nature of the Raman effect.

The intensities are directly proportional to the concentration, unlike infrared spectroscopy, for which Beer's law must be applied. Thus, quantitative analysis by Raman spectroscopy is often easier and more precise.

These advantages are however counterbalanced by the difficulty caused by a few phenomena such as:
Blackbody emission (by heating of the sample);
Fluorescence which is much more intense than the Raman effect when it occurs, but which can be avoided by changing the wavelength;
Photochemical reactions (decomposition of colored substances, etc.);

Multiphoton reactions;

Decomposition of samples by heating.

However, technological progress is tending to reduce the excessive cost of Raman spectroscopy and increase the performance thereof. By way of example, the Fourier Transform Raman spectrometer has the following known additional advantages:
The problem of fluorescence no longer arises by virtue of the use of a low-energy exciting line. Indeed, in Fourier Transform Raman spectroscopy use is made of exciting radiation in the near infrared. This makes it possible to avoid problems associated with the fluorescence of the sample, which may arise when use is made of lasers that operate in the region of visible radiation.
Colored substances are not at risk of decomposing, for the reason given above;
The analysis is more rapid because all the spectral elements are measured simultaneously;
The sensitivity is better because a larger amount of photons are detected simultaneously;
The frequencies are detected with precision by virtue of a laser which measures the position of the movable mirror of the interferometer.

In an article entitled “In vivo blood glucose quantification using Raman spectroscopy”, published in October in the review PLOS ONE volume 7 issue 10 e48127, Jingwei Shao and her team proposed a method of Raman spectroscopy which enables the in vivo detection of plasma glucose concentration. This method is advantageous for the reasons mentioned above, however it is limited to plasma glucose concentration.

No method or apparatus enabling in vivo detection of an exogenous substance, for instance at least one antibiotic, is known.

The U.S. Pat. No. 7,972,296 B2 is known, which describes a method and a system for the dosing and controlled introduction of glucose in the event of glycemia, based on spectrophotometry or mid-infrared spectrometry. Here, glucose or insulin is injected using pouches or a syringe pump. Means able to measure at least one physiological parameter or an analyte are used. Thus, an endogenous substance (glucose or equivalent) is administered into an endogenous liquid (patient's blood).

### DESCRIPTION OF THE INVENTION

The invention aims to overcome the drawbacks of the prior art and especially to propose a device suitable for the detection, measurement and dosing of at least one exogenous substance into an endogenous liquid.

To this end, what is proposed according to a first aspect of the invention is a (portable) device for recognizing, measuring and dosing at least one exogenous substance into an endogenous liquid, comprising: —a means for suction of an endogenous liquid containing at least one exogenous substance; —a means for collection of said endogenous liquid; —a Raman or infrared spectrometer, able to acquire and analyze said collected liquid; —a data storage means of database type comprising spectra relating to at least one exogenous substance; —a means for comparison between the spectrum or spectra acquired and the spectrum or spectra of the database; —at least one means for controlled introduction of at least one exogenous substance into the endogenous liquid, as a function of the result of said spectral comparison.

Preferably, said spectrometer is of Raman or infrared type.

By way of illustration, the exogenous liquid comprises at least one antibiotic while the endogenous liquid consists of blood from a patient. Without departing from the scope of the invention, the exogenous liquid may comprise chemotherapy agents. Other compounds may of course be controlled and monitored (in the common medical sense) in real time; various molecules present in bodily fluids may be identified and quantified in real time, for example cells of the immune system.

The device according to the invention may further comprise at least one means for display of the measurement and/or of the dose and/or of the nature of at least one exogenous substance contained in the endogenous liquid. Any screen, whether touchscreen or not, may constitute the display means.

According to a preferred embodiment of the invention, the mean(s) for controlled introduction enables a variable and controlled rate of introduction of the exogenous substance.

Advantageously, the mean(s) for controlled introduction may be controlled by the Raman or infrared spectrometer.

Advantageously, the device according to the invention may further comprise a device able to vary the temperature of the exogenous substance in its medium, in order to stabilize the physical properties thereof.

According to another feature of the invention, said at least one means for controlled introduction enables monitoring over time and/or in terms of amount of the exogenous substance.

Moreover, said Raman or infrared spectra are correlated to variations in temperature and/or to variations in acidity of the endogenous liquid.

In addition, the device according to the invention may comprise means for centrifuging the endogenous liquid.

According to a particular embodiment of the invention, the device comprises several means for controlled introduction of an exogenous substance, mounted on a carrier structure, and each being connected to the means for comparison and computation.

The invention also targets a process for modifying the composition of an endogenous liquid by an exogenous substance, which especially comprises the following steps: —collection of the endogenous liquid at the inlet of a device as described; —analysis of said liquid by spectrometry, preferably Raman or infrared spectrometry; —storage of spectra in a database, said spectra relating to at least one exogenous substance; —comparison of at least one stored spectrum with at least one reference spectrum of an element of the endogenous liquid; —rate-controlled introduction of at least one exogenous substance into the endogenous liquid emerging from the device, as a function of the result of said spectral comparison.

### BRIEF DESCRIPTION OF THE FIGURES

Other features, details and advantages of the invention will emerge from reading the following description with reference to the appended figures which illustrate:

FIG. 1: an outer view of the device according to an embodiment of the invention;

FIG. 2: a flowchart of the operating principle of a Raman spectrometer;

FIG. 3: Raman spectra for three exogenous substances of antibiotic type;

FIG. 4: an example of Raman spectrum obtained for two different exogenous substances; and

FIG. 5: a perspective view of a particular embodiment of the invention.

For greater clarity, identical or similar elements are identified with identical reference signs across all the figures.

### DETAILED DESCRIPTION OF AN EMBODIMENT

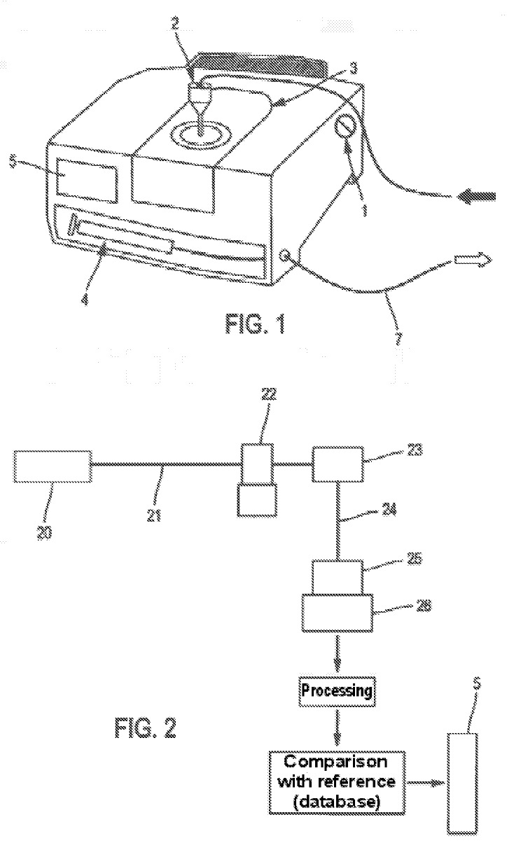

FIG. 1 illustrates a device in accordance with the invention; more specifically, the device comprises a means for suction 1 of an endogenous fluid such as blood. This suction means 1 is connected to a collector 2 which makes it possible to collect a determined amount (or sample) of the endogenous fluid. The collected sample is analyzed by a Raman spectrometer 3 preferentially arranged in the device 1 in order to form a dedicated autonomous assembly. Of course, all the connections and other appropriate links are provided so that the collected fluid can be analyzed by the Raman spectrometer. These connections and links will not be described in detail because they are within the scope of those skilled in the art.

The device further comprises a means for controlled introduction of the exogenous fluid, either directly into the endogenous fluid while it circulates in the device itself, or in a separate circuit. In any case, it is a question of dosing and therefore of controlling the exogenous fluid which must be introduced into the endogenous fluid or into the body of a patient. For this purpose, a means of syringe pump type 4 is used. The method used will be described below. Advantageously, the operation of the syringe pump is controlled by the Raman spectrometer, as will be explained below.

Moreover, the device according to the invention is preferentially provided with a means for display 5 of the measurement and/or of the amount and/or of the dose and/or of the nature of at least one exogenous substance present in the endogenous liquid. The display means may be a screen, whether touchscreen or not.

Preferably, and for the advantages mentioned above, a Raman spectrometer is used.

FIG. 2 illustrates the operating principle of the Raman spectrometer in the context of the invention. Given that, in a preferred application of the invention, it is blood plasma which is targeted as endogenous liquid, it appears that the Raman spectrometer is, in this application case, the most suitable means for carrying out the invention. This is because it is known that water constitutes more than 90% of blood plasma, hence the choice of the Raman spectrometer. Without departing from the scope of the invention, an infrared spectrometer may be used.

According to FIG. 2, the radiation from a laser source 20 is conveyed through an optical fiber 21 which passes through the collected sample 22 to be analyzed. This then creates heating in the sample, the light produced is sensed by a sensor 23 and conveyed by an optical fiber 24 to a separator 25 coupled to a detector 26 which provides data relating to the collected sample 22. This data is then processed by a suitable computer system, not depicted structurally. The data consists of spectra.

The processing of the data consists of a comparison between the spectra relating to the data relating to the collected sample and the spectra originating from a database, stored in the computer system. The result of this comparison may take different forms which may be displayed on the means 5.

It is a question here of configuring a Raman spectrometer in order to determine, virtually in real time, the dosage, the concentration of the exogenous substance in the endogenous liquid leaving the device according to the invention.

As already mentioned briefly, the operation of the syringe pump 4 depends in particular on the results of the comparison between the known spectra and the spectra of the collected liquid. An electric syringe pump may thus be chosen for this functionality. More specifically, the syringe pump 4 may advantageously modify the rate of perfusion of the exogenous substance as a function of the blood concentration measured by the device according to the invention. Thus, regardless of the other parameters, the amount of exogenous substance administered is constant.

An adaptation of the rate of perfusion is carried out according to pre-established and selected objectives. In the case of application of an antibiotic therapy, the prescription constitutes the basis of the pre-established objectives.

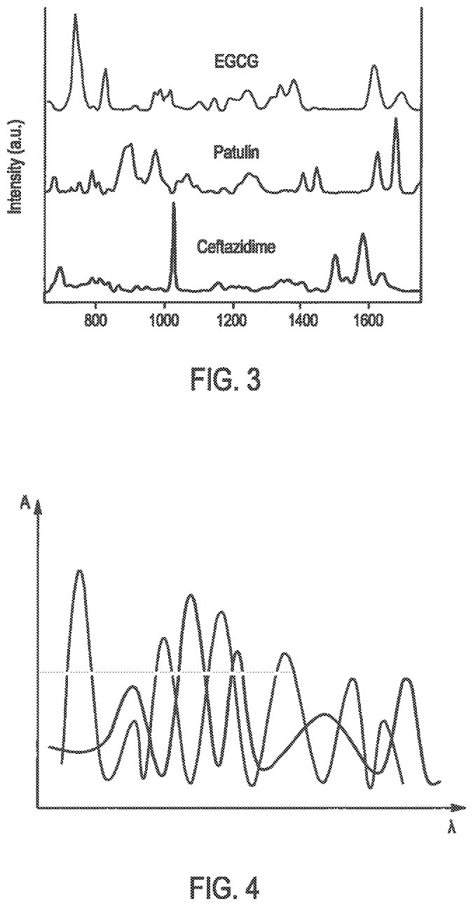

As shown in FIG. 3, each antibiotic has a specific spectrum, that is to say a characteristic signature or fingerprint. In a known way, FIG. 3 gives curves of intensity as a function of the wavenumber (in cm⁻¹). FIG. 3 gives three spectra defined by intensity as a function of the Raman shift (intensity number); the 3 antibiotics illustrated correspond respectively to EGCG (top curve), Patulin (middle curve) and Ceftazidime (bottom curve).

It is therefore necessary to form a database consisting of Raman spectra for different exogenous substances likely to be collected for analysis within the context of the invention.

Regarding the development of a database, illustration is given here of the case of developing a database in the field of antibiotics. Indeed, in antibiotic therapy it is crucial to evaluate the therapeutic concentrations specific to each antibiotic; the aim being to know the range of concentrations necessary for the respective calibration curve for each antibiotic.

Advantageously, in the case of measuring and dosing antibiotics in blood, the database takes into account variations in temperature and acidity of the blood in order to interpret the plasma concentrations of antibiotics.

Each antibiotic (from a pure solution) will be analyzed by Raman spectroscopy, for a range of concentrations defined beforehand. In order to take into account uncontrolled variations inherent to the experiment, it is necessary to accumulate spectra from numerous samples. For the purposes of reliability, these analyses should therefore be repeated several times under the same conditions. FIG. 4 illustrates the results of such an accumulation of spectra. The temperature represents another parameter to be included. Indeed, it is essential that the analysis is carried out at different temperatures (from 34° C.+/−0.1 to 41° C.). This would subsequently make it possible to ensure the reliability of the data, by taking into account the body temperature of the patient.

Moreover, a step of calibration of the data is essential before any routine use. The use of a calibration curve makes it possible to solve all the problems associated with non-linearity of the absorbance curve or the effective concentrations of absorbent entities; this means that any measurement for a solution with an unknown titer, carried out under the same conditions as for a series of standards, makes it possible to directly draw conclusions from the curve. The recommended method requires that the measurements for all the standards and all the samples are carried out in the same cell, with the same optical path, but it is not necessary to know either the exact dimensions of cell or the molar attenuation coefficient for the absorption band chosen, since these are constants. The spectral collection thus obtained will be processed by chemometric methods. However, before being utilized, the raw spectral data will be subject to various pre-processing operations, the aims of which are:
attenuation or elimination of the non-linearity present between dependent and explanatory variables,
elimination of interference,
elimination or attenuation of the random noise associated with the experimental conditions and the electronic noise of the measurement apparatus,
reduction of correlations between the explanatory variables to enable the application of multiple linear regression techniques.

The spectra collected may be represented in the form of a matrix. This matrix representation thus makes it possible to carry out multivariate analyses. Where n is the samples analyzed by a Raman spectrometer and p is the number of different wavelengths measured by the instrument. The spectral matrix n×p has the following form:

$$X = ( \begin{matrix}{I}_{1}^{1} & … & {I}_{1}^{p} \\ ⋮ & ⋱ & ⋮ \\ {I}_{n}^{1} & … & {I}_{n}^{p}\end{matrix} )$$

Each row corresponds to a sample and each column represents the absorbances of the samples for a given wavelength.

The most common strategy consists in subjecting the raw data to one or more mathematical transformation(s) intended to make them suitable for linear modelling. The choice of the transformation depends on the type of non-linearity affecting the data and therefore on the quality of the predictive models developed for each pre-processing operation.

A quantitative method by multivariate analyses is carried out, which comprises a regression analysis on samples of known concentrations, and a prediction for unknown samples from analyses of samples of unknown concentrations.

It is subsequently essential to have parameters making it possible to validate the adjustment of a model and/or to compare several models in order to eventually choose the model which proposes the best results in terms of prediction.

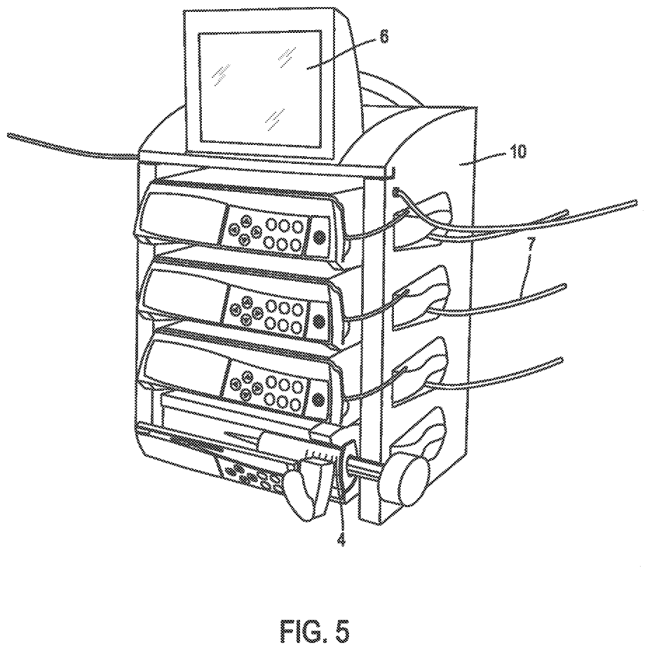

FIG. 5 illustrates a particular embodiment of the invention which comprises means 4 for controlled introduction of an exogenous substance into the endogenous liquid. The means 4 here are syringe pumps, numbering four, mounted on the same carrier structure 10. The exogenous liquid is introduced by a pipe 7 into each associated means 4. It emerges therefrom by a pipe which cannot be seen in FIG. 5, at a rate determined by the means for comparison and computation. A general display means 6 may be provided in order to view the different parameters and calculation results.

Although the above description is based on an exogenous substance of antibiotic type, the device and the process according to the invention can be applied and used with any type of exogenous substance. In particular, the exogenous substance may be an anti-viral, an antifungal, or generally speaking an antibacterial agent. The exogenous substance may also be a substance used in chemotherapy.

Of course, the device and the process according to the invention are not limited to the application cases illustrated above; the invention enables any real-time analysis of an exogenous substance in an endogenous liquid associated with an introduction and/or an injection controlled by the results of the analysis.

1.-10. (canceled)

11. A device for recognizing, measuring and dosing at least one exogenous substance into an endogenous liquid, comprising:
a means for suction of an endogenous liquid containing at least one exogenous substance;
a collector for the endogenous liquid;
a spectrometer configured to acquire and analyze the collected endogenous liquid;
a processor containing a database comprising spectra relating to at least one exogenous substance and configured to compare spectrum or spectra acquired by the spectrometer and the spectrum or spectra in the database;
at least one pump for controlled introduction of at least one exogenous substance into the endogenous liquid, as a function of the result of the spectral comparison.

12. The device according to claim 1, wherein the spectrometer is a Raman or infrared spectrometer.

13. The device according to claim 1 further comprising a display of a measurement of, a dose of, a nature of at least one exogenous substance contained in the endogenous liquid.

14. The device according to claim 1 wherein the pump if configured to provide a variable and controlled rate of introduction of the exogenous substance.

15. The device according to claim 1 wherein the pump is controlled by the spectrometer.

16. The device according to claim 1 further comprising a device configured to vary the temperature of the exogenous substance.

17. The device according to claim 1 wherein the pump enables monitoring over time and/or in terms of amount of the exogenous substance.

18. The device according to claim 1 wherein the stored spectra are correlated to variations in temperature and/or to variations in acidity of the endogenous liquid.

19. The device according to claim 1 further comprising a centrifuge for centrifuging the endogenous liquid.

20. The device according to claim 1 further comprising additional pumps for controlled introduction of an exogenous substance, at least one of which is mounted on a carrier structure and each being connected to the processor.

21. The device according to claim 1 wherein the exogenous liquid comprises at least one antibiotic.

22. A process for modifying the composition of an endogenous liquid by an exogenous substance comprising:
sucking an endogenous liquid from a mammal into a device;
collecting the liquid;
analyzing the liquid using spectrometry to obtain a spectra relating to at least one measured exogenous substance;
comparing the obtained spectra to at least one stored spectrum with at least one reference spectrum of an element of the endogenous liquid;
introducing at least one exogenous substance in a rate-controlled manner into the endogenous liquid emerging from the device, as a function of the result of the spectral comparison.
